(19) United States
(12) Patent Application Publication
(10) Pub. No.: US 2004/0092004 A1
(43) Pub. Date: May 13, 2004

(54) Sensor for detection of enzyme and enzyme detection method for aerosolized bacteria in the enviromnment

(75) Inventors: Thomas B. Stanford JR., Hueneme, CA (US); Camille I. Van Ast, Newbury Park, CA (US)

Correspondence Address: Richard P. Berg, Esq., c/o LADAS & PARRY, Suite 2100, 5670 Wilshire Boulevard, Los Angeles, CA, 90036-5679, US

(73) Assignee: HRL Laboratories, LLC

(21) Appl. No.: 10/680,937

(22) Filed: Oct. 7, 2003

Related U.S. Application Data
(60) Provisional application No. 60/425,039, filed on Nov. 7, 2002.

(51) Int. Cl. C12M 1/34; C12Q 1/40

(52) U.S. Cl. 435/287.1; 435/22

(57) ABSTRACT

A sensor for the detection of organisms and a method for the detection of organisms is provided, the sensor comprises a substrate; at least one pair of electrodes; a sol gel matrix comprising at least one enzyme; at least one reactant; and at least one transducer material; wherein (a) an organism expresses an enzyme on the surface of the sensor; (b) the enzyme catalyzes a reaction of the reactant of the sensor; (c) the product according to process step (b) catalytically reacted by said enzyme of the sensor; (d) the products of process step (c) modulate at least one property of the transducer material; and (e) the modulated property is measured.

### CROSS REFERENCE TO PRIOR APPLICATION

This application claims the benefits of U.S. Provisional Patent Application serial No. 60/425,039 filed Nov. 7, 2002, the disclosure of which is hereby incorporated by reference.

### BACKGROUND OF THE INVENTION

1. Field of the Invention

This invention relates to a device and method for the detection of the presence of bacteria, viruses, and/or other organisms by detecting metabolic and other processes associated with such organisms instead of the organisms themselves. The development of sensors that can detect biological warfare agents (BWAs) is critically needed by government agencies, such as the Department of Justice, the Department of Defense, and civilian first-response teams.

2. Description of the Related Art

The bioindicators used in typical prior art detection concepts for bacteria, viruses, and other organisms are highly specific for the target organism and can provide in some cases, quantification of a target organism. As target organisms and biomaterials become more complex, indicators for such organisms similarly become more exotic, and are often expensive, difficult to handle and in some cases unavailable. This handicap frequently limits or precludes the application of this approach for the detection of different bio-organisms. For the detection of bacteria and viruses, bioindicators such as antibodies, macrophages, and/or phagocytes are typically immobilized on the transducer and their interaction(s) with the target species is monitored using a variety of techniques (i.e. optical, electrical, gravimetrical, etc.). However, although highly specific, most such bioindicators are quite labile and easily denatured by harsh or incompatible environmental conditions.

Prior sensors used in biodetection concepts have sought to exploit the highly selective and specific interactions that occur, for example, between bacteria and antibodies or bacteria and bacteriophages. Antibodies are created by an immune system to specifically target an invading organism. Phages are designed to infect only a specifically coded bacterial host. This traditional sensor is therefore selective for a single process/interaction that is associated with an organism. Such interactions or processes selected for monitoring are therefore highly selective but limited in number.

U.S. Pat. No. 5,417,100 discloses a sensor for detecting volatile hydrocarbons and other resolvent vapors which detects leaks in the fittings and valves of petroleum refineries and chemical manufacturing and processing plants. The sensor comprises a dielectric substrate having a major surface; a pair of interdigitated, electrically conductive electrodes disposed on the major surface of the substrate; and a composite coating covering the interdigitated electrodes and comprising a conductive polymer, and a dielectric polymer with an affinity for the solvent vapors to be detected.

The composite coating contains a conductive polymer which is blended in a non-conductive polymer host. Volatile hydrocarbons contained in the vapor are absorbed by the host polymer causing it to swell, thereby increasing the relative separation of the conductive polymer species embedded therein. A change of the relative position of the embedded conductive polymer molecules results in a change of their conductivity.

U.S. Pat. No. 5,756,879 discloses a sensor and method for detecting volatile compounds in the gas phase at concentrations of less than about 500 ppm in ambient air. The sensor comprises a dielectric substrate having a major surface; a pair of electrically conductive electrodes disposed on the major surface of the substrate; and a conductive polymer covering the pair of electrically conductive electrodes, with the conductive polymer doped with appropriate dopants in measurable excess of that stoichiometrically required to change the conductive polymer from a neutral state to a charged state to provide requisite conductivity.

U.S. Pat. No. 5,756,879 further discloses the use of the sensor, which includes the detection of fugitive emissions in chemical plant environments; the detection of certain pollutants in vehicle exhaust; and the detection of certain pollutants near chemical handling operations, such as painting operations.

The sensor contains a conductive polymer material. Volatile compounds influence the change of the conductivity of the conductive polymer element by chemical interaction by exchanging dopant ions.

One such technique is Time-of-Flight (TOF) mass spectrometry. With TOF mass spectrometry, the sample, usually several hundred μgrams, must be collected prior to analysis, then decomposed by heating and the fragments analyzed. This technology is not passive or real-time and requires high power and complex, expensive instrumentation and data analysis.

A gravimetric technique and resultant device called aerogel-SAW, uses a biospecific indicator, but requires the formation of a stable, non-volatile intermediate or by-product of the interaction of the bioindicator and the target bacteria or virus. This technique, and resultant device is therefore much less versatile and typically less sensitive than the current invention and requires more complex electronics.

A micro-device for collection and separation of biosamples from environmental interferences is called a μChem Lab or Lab-on-a-chip. This type of sensor is amenable to multiple detection concepts, but burdened with formidable plumbing difficulties such as valve operation, plugged lines and manifolds.

Another prior art technology is DNA analysis. This type of analysis detects bacteria-specific DNA sequences based on Polymerase Chain Reaction (PCR). This type of analysis allows for high sensitivity. The disadvantages of this device are that it requires prior knowledge of target pathogens, is not real-time or passive and requires complex equipment and procedures.

Yet another prior art technique for detecting organisms involves fluorescence quenching. This technique is antibody based, highly specific, sensitive, useful for both bacteria and viruses. However, this technique requires foreknowledge of the target pathogen and utilizes antibodies that are often unstable or difficult to find. What is needed is a device, a sensor for detection of enzyme and enzyme detection method which do not require prior knowledge of the target pathogen.

### SUMMARY

Typically, the sensors disclosed herein are based on the interaction of an enzyme of a bacteria or virus with an appropriately selected bioindicator. The bioindicator is then monitored for modulation of some property, which is indicative of the desired interaction. All bacteria and many viruses have associated enzymes, many of which are exogenic. Bacteria and viruses must utilize certain specific enzymes for the performance of necessary functions such as cell wall synthesis, hemolysis, H₂O₂ reduction, and numerous metabolic processes which are characteristic of these species.

The sensors determine the presence and identity of bacteria and/or viruses by detecting the enzymes associated with these organisms. Bacteria are characterized by the particular combination of enzymes which are necessary for critical metabolic processes. The enzymes detected are responsible for the performance of reactive processes essential to the organism, such as, but not limited to, starch hydrolysis, glycolysis, anaerobic respiration, denitrification, and/or triglyceride hydrolysis. In accordance with the present invention a sensor is provided which comprises

(a) a substrate;

(b) at least one pair of electrodes;

(c) an encapsulating matrix comprising;

(d) at least one enzyme;

(e) at least one reactant; and

(f) at least one transducer material.

An embodiment of the current invention is a sensor for detecting bacteria, viruses and other organisms. The sensor comprises a reactant disposed within said encapsulating matrix, a second enzyme encapsulated in said encapsulating matrix, a transducer material embedded within said encapsulating matrix and an instrument to measure an electric current flowing through said electrodes. The encapsulating matrix can be a sol gel matrix according to the present invention. However, other encapsulating gels may be employed.

The sensor of this disclosure is specific not for the target organism, but rather for discrete by-products of the reactive processes which are conducted by that organism. The sensor of the current invention interrogates the target bacteria, viruses, or other organisms by monitoring for selected reactive processes and associated by-products, which are conducted within the sensor. The by-products of these reactive processes are then detected by their ability to modulate conductivity of an inherently conductive polymer also imbedded in the sensor. As the suite of enzymatic processes conducted by an organism are characteristic of that organism, this invention is a powerful tool for identification and discrimination of such organisms.

Further, in accordance with the present invention a method is provided by a sensor comprising a substrate; at least one pair of electrodes; an encapsulating matrix comprising at least one enzyme; at least one reactant; and at least one transducer material; wherein

(a) an organism expresses an enzyme on the surface of the sensor;

(b) the enzyme reacts with the reactant of the sensor;

(c) the product according to process step (b) reacts with said enzyme of the sensor;

(d) the products of process step (c) modulate at least one property of the transducer material;

(e) the modulated property is measured.

An embodiment of the current invention is a method for the detection of bacteria, viruses, and other organisms comprising disposing a transducer material within said encapsulating matrix, disposing a reactant in said encapsulating matrix. Said reactant reacts with an enzyme expressed by said bacteria, viruses, or other organism to form a product. An enzyme is encapsulated in said encapsulating matrix. Said product reacts with said enzyme to form other product. Said transducer material indicates the presence of said other product. An electric voltage is applied to said electrodes. A change in an electric current flowing through said electrodes is measured. Said change is caused by an interaction of said transducer material with said other product being a consequence of the presence of said bacteria, viruses, or other organisms.

The disclosed method is particularly advantageous for environmental detection methods, which typically require inexpensive, compact devices that are easily handled and have long shelf lives and extended field use. The device of this invention utilizes materials that are much less complex and more easily handled, readily obtained, and more durable to environmental conditions than typical bioindicators for bacteria, viruses, or other organisms. Concepts based on prior art devices require multi-step analyses, which call for labile and expensive reagents and skilled operators to perform and interpret test results. The concept of this invention will greatly simplify the detection devices that may be used for detection of bacteria, viruses, or other organisms including airborne organisms.

### DRAWINGS

FIG. 1 depicts a preferred embodiment of the device according to the disclosure.

FIG. 1a depicts a cross-section of the preferred embodiment of the device depicted in FIG. 1 along the line A-A.

FIG. 2 helps explain a preferred embodiment of the method of the present invention.

### DESCRIPTION

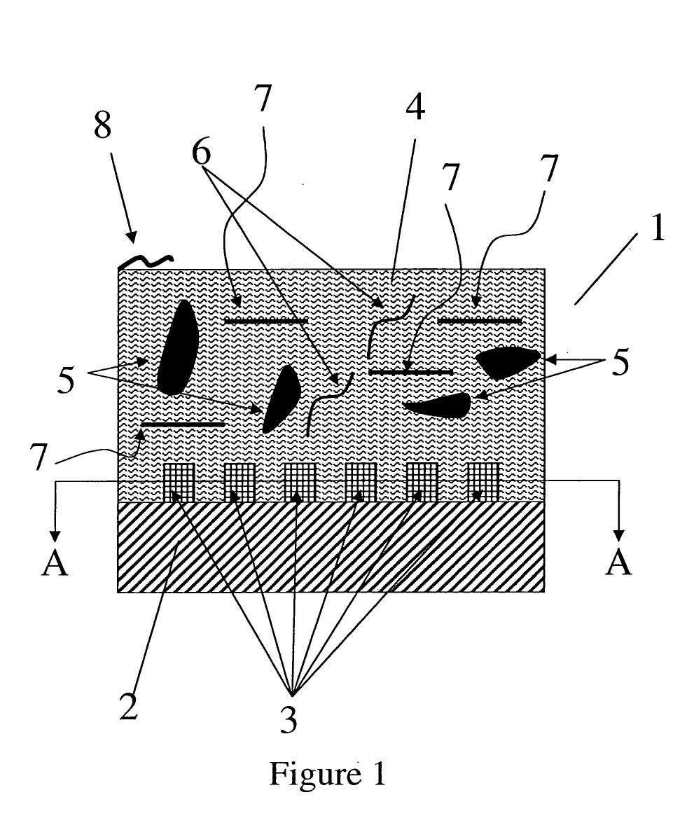

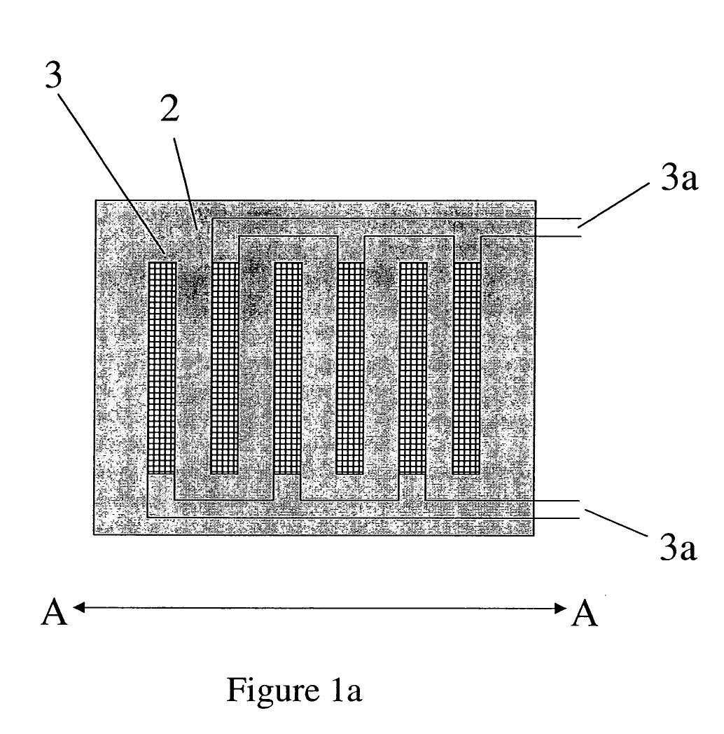

The preferred method for detecting bacteria and other organisms is described in detail below. In a preferred embodiment of the device 1 of the current invention, shown in FIG. 1, a substrate 2, preferably glass, is provided with at least one pair of interdigitated electrodes 3. The electrodes 3 are preferably made of gold due to gold's high electrical conductivity and chemically inert nature. However, other highly conductive metals such as platinum, silver, or copper may be suitable. Modulation of the conductivity, upon which detection of pathogens depend, is accomplished by a chemical reaction and subsequent modification of the material.

The shape of the electrodes 3 is preferably rectangular in shape and cross section. However, other designs may be employed such as circular shape and/or elliptical cross-section. Each electrode 3 comprises a plurality of digits, the digits interleaving. The width of each gold digit is within a range of between about 5 micrometers and about 25 micrometers, preferably about 15 micrometers. The gaps between the digits are within a range of between about 5 micrometers and about 25 micrometers, preferably about 15 micrometers. The thickness of each digit is within a range of about 1 micrometer to about 4 micrometers. About 30 to 60, preferably 40 to 50 line pairs of digits are preferably used, but the number of such line pairs can vary with the application and the dimensions of the sensor element required for a particular application.

A sol gel matrix 4, shown in FIG. 1, used as an encapsulant, is disposed over the electrodes 3 and substrate 2. The sol-gel matrix has many distinct advantages. However, other encapsulating gels may be employed. Enzymes can be readily encapsulated within the cavities of a sol-gel matrix by relatively simple methods. As shown in FIG. 1, embedded within the sol gel matrix 4, is an enzyme 5, which is capable of detecting the presence of a product or products from reactive processes associated with bacteria, viruses, or other organisms. Since these enzymes 5 can be chosen for their interaction or reaction with only their conjugate, specificity is rendered to the sensor even in the presence of other possibly interfering pollutants or pathogens.

For those enzymes that have an intrinsic steric conformation (i.e., coiled structure) that determines their biological activity, their stability can become compromised with increasing temperature resulting in a modification of their conformation (i.e., uncoiling or denaturation) and therefore a loss in activity. Encapsulation of these enzymes in a sol-gel matrix, however, precludes this process since the molecules are confined within the cell of the sol-gel making it more difficult to denature. This results in enhanced thermal stability. Further, these molecules require an aqueous environment for their viability. During the encapsulation process, water is also captured with the enzyme, which then also enhances the stability of the enzymes. The resulting sol-gel film, however, is dry to the touch. Thus, an external supply of aqueous medium is not required to retain the viability of the enzyme, which greatly reduces the complexity of the resulting sensor element. Furthermore, these enzymes require the proper pH for their viability. The sol-gel is also used to encapsulate the proper buffer materials to generate the required pH environment about the enzyme.

Very close contact between the enzyme component and the sol-gel derived material component is very desirable and is achieved by bringing these two components into such close contact through dispersing of the enzyme within the matrix formed by the sol-gel derived material component. Using a single thin composite film where the enzyme component and the sol-gel derived material component are in such close contact is preferred.

FIG. 1 shows a preferred embodiment of the sensor containing a substrate 2 and at least one pair of electrodes 3. A transducer material 6 is dispersed within the sol gel matrix 4. Organosilanes may be used in the sol gel formulations for the matrix 4. The organosilanes may be tetrafunctional like tetramethoxy orthosilicate, trifunctional, like methyltrimethoxysilane, octadecyltrichlorosilane, octadecyltriethoxysilane, phenyltrimethoxysilane and 1,4-bis(trimethoxysilylethyl)benzene, or difunctional, like methyldimethoxysilane, dimethyldiethoxysilane, or monofunctional, like octadecyldimethylmethoxysilane, or derivatized silanes, like 2-(3,4-epoxycyclohexyl)ethyltrimethoxysilane, 3-aminopropyltrimethoxysilane, 4-aminobutyldimethoxysilane, N-(2-aminoethyl)-3-aminopropylmethyldimethoxysilane, 5-(bicycloheptenyl)triethoxysilane, dicyclohexyldimethoxysilane and 3-glycidylpropyltrimethoxysilane. The enzyme 5 may be one selected from the group including but not limited to tryptophanase, gelatinase, β-lactamase, catalase, casease, citrase, decarboxylase, deoxyribonuclease, lipase, nitrate reductase, β-galactosidase, cytochrome oxidase, phenylalanine deaminase, 1-pyrrolidonyl arylamidase, cystein desulfase, urease, L-asparaginase, glutamate dehydrogenase, organphosphorus hydrolase, acetylcholinesterase, α-amylase and glucose oxidase. The transducer material 6 is preferably a water-soluble polymer including, but not limited to, polyanilines, polythiophenes, polysulphonic acids, derivatives thereof, and combinations thereof. Furthermore, dispersed in the sol gel matrix 4 is a reactant 7. In a preferred embodiment, the enzyme 5 embedded within the sol gel matrix 4, is glucose oxidase, and the reactant 7 is preferably a starch containing amylose.

The matrix 4 containing the enzyme 5, transducer material 6 and the reactant 7 are preferably in the following concentration.

| Component | Concentration | No. |
|---|---|---|
| SOL GEL | from about 0.1 M to about 1 M | 4 |
| ENZYME | from about 0.01% to about 1.0% by weight | 5 |
| TRANSDUCER | from about 1 * 10⁻³ M to about 1 * 10⁻⁵ M | 6 |
| MATERIAL | | |
| REACTANT | from about 0.01% to about 1.0% by weight | 7 |

A preferred content of the matrix is as follows:

| | Concen- | Compo- | |
|---|---|---|---|
| Compound | tration | nent | No. |
| TETRAMETHOXY ORTHOSILICATE | 0.21 M | siloxane | 4 |
| (TMOS) | | | |
| GLYCIDYLPROPYLTRIMETHOXYSILANE | 0.21 M | siloxane | 4 |
| (GPTMS) | | | |
| GLUCOSE OXIDASE (GOₓ) | 0.29% | enzyme | 5 |
| | by | | |
| | weight | | |
| POLY (ANILINESULFONIC ACID) | 8.2 * | trans- | 6 |
| (PAS) | 10⁻⁴ M | ducer | |
| | | material | |
| AMYLOSE | 0.25% | starch | 7 |
| | by | material | |
| | weight | | |

FIG. 1a shows a cross section of the device of FIG. 1 along the line A-A. It shows the substrate 2 of the device 1 and a cross section of the electrodes 3. As it can be seen, the electrodes 3 are connected by wires 3a as an interdigitated structure.

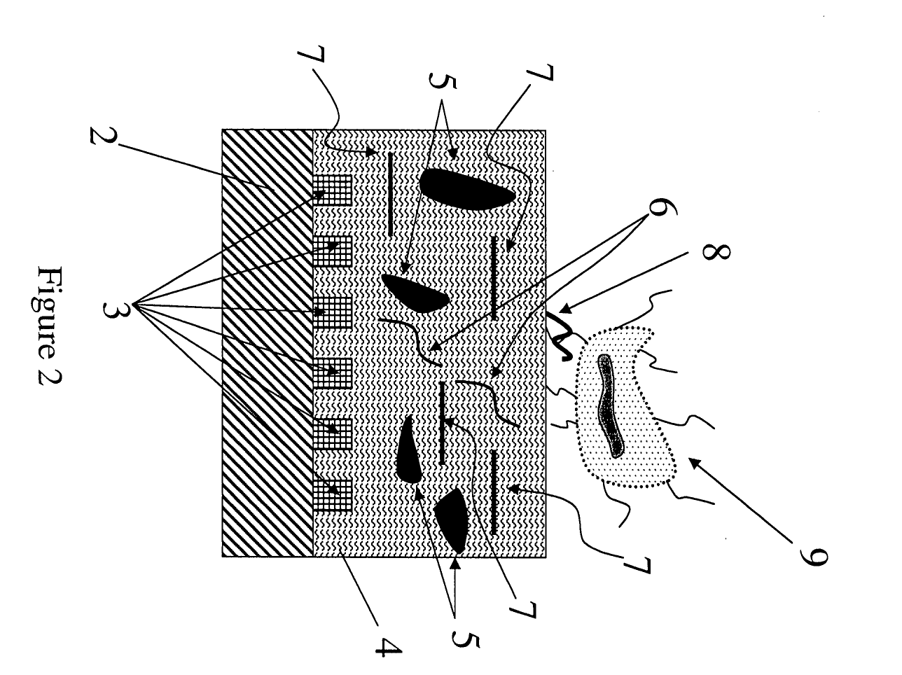

FIG. 2 helps explain a preferred embodiment of the method of the disclosure as will be shown by the disclosed examples. According to the preferred method, bacteria, viruses, or other organisms 9, which can be airborne, come into contact with the sol gel matrix 4. The bacteria, viruses, or other organisms express an enzyme 8. The expressed enzyme 8 may be one selected from the group consisting of tryptophanase, gelatinase, β-lactamase, catalase, casease, citrase, decarboxylase, deoxyribonuclease, lipase, nitrate reductase, β-galactosidase, cytochrome oxidase, phenylalanine deaminase, 1-pyrrolidonyl arylamidase, cystein desulfase, urease, L-asparaginase, glutamate dehydrogenase, organphosphorus hydrolase, acetylcholinesterase, and α-amylase.

The expressed enzyme 8 is preferably α-amylase. The reactant 7, preferably a starch containing amylose, reacts with the enzyme 8 α-amylase. Enzyme 8, α-amylase catalyzes the hydrolysis of the amylose contained in the reactant starch 7. The sol gel matrix 4 forms a protective cavity around enzyme 8 to maintain the enzyme's viability, while the transducer material 6 is dispersed in close proximity in the surrounding sol gel matrix 4. Polymerizing the sol gel matrix 4 around the enzyme 5 embeds the enzyme 5.

### EXAMPLES

### Example 1

The bacteria, virus, or other organism 9 expresses an enzyme 8. The enzyme 8 causes the reaction of the reactant 7 to form a product as shown in reaction scheme I.

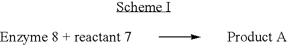

Then Product A reacts through enzyme 5 to form other products B or products B as shown in reaction scheme II.

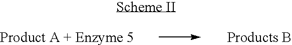

These final products B or product B modulate some property of transducer material 6 and are detectable by measuring means as shown in reaction scheme III.

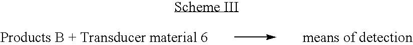

An exemplary example of a preferred embodiment is as follows.

### Example 2

The enzyme 8, expressed by the bacteria, virus, or other organism 9, is α-amylase. The α-amylase 8 causes the hydrolysis of the starch 7 containing amylose to form glucose A as shown in reaction scheme Ia below.

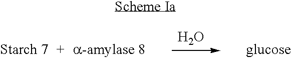

The glucose A is catalytically oxidized by glucose oxidase 5 to form other products B, namely gluconic acid and H₂O₂ shown in reaction scheme IIa.

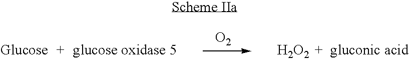

The gluconic acid and H₂O₂ are sensed by the transducer material 6, or conductive polymer 6. The products formed from the oxidation of glucose catalyzed by enzyme 5, namely gluconic acid and H₂O₂, modulate the electrical resistance of an inherently conductive polymer 6, or transducer material 6, as shown in reaction scheme IIIa.

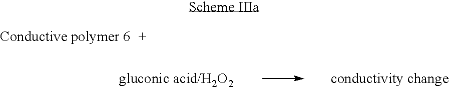

The electrodes, along with a voltage source and ohmmeter, are used to probe the modulated electrical resistance of the inherently conductive polymer.

Those skilled in the art will recognize, or be able to ascertain employing no more than routine experimentation many equivalents to the specific structures, steps, functions, and materials described specifically herein, and such equivalents are intended to be encompassed within the scope of the following claims. Inclusion of compositions and any other features related to any materials disclosed herein is hereby incorporated into the specification by mere reference to these materials.

Let it be understood that the foregoing description is only illustrative of the invention. Various alternatives and modifications can be devised by those skilled in the art without departing from the spirit of the invention. Accordingly, the present invention is intended to embrace all such alternatives, modifications, and variances which fall within the scope of the appended claims.

What is claimed is:

1. A sensor comprising:
(a) a substrate;
(b) at least one pair of electrodes;
(c) an encapsulating matrix comprising;
(d) at least one enzyme;
(e) at least one reactant; and
(f) at least one transducer material.

2. The sensor of claim 1, wherein said substrate is selected from the group consisting of glass, ceramic, and plastic.

3. The sensor of claim 1, wherein said electrodes comprise one or more elements selected from the group consisting of gold, platinum, or silver.

4. The sensor of claim 1, wherein said electrodes are interdigitated.

5. The sensor of claim 1, wherein said sol gel matrix covers the substrate and the electrodes.

6. The sensor of claim 1, wherein said encapsulating matrix is a sol gel, said sol gel matrix comprising at least one organosilane, which may be tetrafunctional, like tetramethoxy orthosilicate, trifunctional, like methyltrimethoxysilane, octadecyltrichlorosilane, octadecyltriethoxysilane, phenyltrimethoxysilane and 1,4-bis(trimethoxysilylethyl)benzene, or difunctional, like methyldimethoxysilane, dimethyldiethoxysilane, or monofunctional, like octadecyldimethylmethoxysilane, or derivatized silanes, like 2-(3,4-epoxycyclohexyl)ethyltrimethoxysilane, 3-aminopropyltrimethoxsilyane, 4-aminobutyldimethoxysilane, N-(2-aminoethyl)-3-aminopropylmethyldimethoxysilane, 5-(bicycloheptenyl)triethoxysilane, dicyclohexyldimethoxysilane and 3-glycidylpropyltrimethoxysilane.

7. The sensor of claim 1, wherein said encapsulating matrix encapsulates said enzyme.

8. The sensor of claim 8, wherein said enzyme may be one selected from the group consisting of tryptophanase, gelatinase, β-lactamase, catalase, casease, citrase, decarboxylase, deoxyribonuclease, lipase, nitrate reductase, β-galactosidase, cytochrome oxidase, phenylalanine deaminase, 1-pyrrolidonyl arylamidase, cystein desulfase, urease, L-asparaginase, glutamate dehydrogenase, organphosphorus hydrolase, acetylcholinesterase, α-amylaseand and is preferably glucose oxidase.

9. The sensor of claim 1, wherein said transducer material and said reactant are dispersed in said encapsulating matrix.

10. The sensor of claim 1, wherein said reactant is starch containing amylose.

11. The sensor of claim 1, wherein said transducer material is a polymer.

12. The sensor of claim 11, wherein said polymer is a water soluble polymer.

13. The sensor of claim 12, wherein said water-soluble polymer is selected from the group consisting of polyaniline, polythiophenes, polyacetylenes, polypyrroles, and combinations thereof.

14. A method for the detection of organisms by a sensor, the sensor comprising a substrate; at least one electrode; a sol gel matrix comprising; at least one enzyme; at least one reactant; and at least one transducer material; wherein
(a) an organism expresses an enzyme on the surface of the sensor;
(b) the enzyme causes a reaction with the reactant of the sensor;
(c) the product according to process step (b) reacts further as catalyzed by said enzyme of the sensor;
(d) the products of process step (c) modulate at least one property of the transducer material;
(e) the modulated property is measured.

15. The method according to claim 14, wherein the expressed enzyme is selected from the group consisting of tryptophanase, gelatinase, β-lactamase, catalase, casease, citrase, decarboxylase, deoxyribonuclease, lipase, nitrate reductase, β-galactosidase, cytochrome oxidase, phenylalanine deaminase, 1-pyrrolidonyl arylamidase, cystein desulfase, urease, L-asparaginase, glutamate dehydrogenase, organphosphorus hydrolase, acetylcholinesterase, and α-amylase.

16. The method of claim 14, wherein said product of process step (c) is selected from the group consisting of lactic acid, carbon dioxide, hydrogen, ethanol, acetic acid, succinic acid, gluconic acid, and formic acid.

17. The method according to claim 14, wherein
(a) an organism expresses α-amylase;
(b) α-amylase catalyzes the hydrolysis of starch to form glucose;
(c) glucose oxidation is catalyzed by glucose oxidase to form gluconic acid and H₂O₂;
(d) gluconic acid and H₂O₂ modulate the electrical resistance of an inherently conductive polymer, or transducer material;
(e) the modulated electrical resistance of the inherently conductive polymer, or transducer material is measured with a voltage source and ohmmeter.
